(19) United States
(12) Patent Application Publication
(10) Pub. No.: US 2024/0342834 A1
(43) Pub. Date: Oct. 17, 2024

(54) Apparatus of Inner Light Layer Formed by Multibeam Interference for Processing Object in Optical Turbid Medium

(71) Applicants: Joyce Liu, Rochester, NY (US); Shangqing Liu, Toronto (CA)

(72) Inventors: Joyce Liu, Rochester, NY (US); Shangqing Liu, Toronto (CA)

(21) Appl. No.: 18/594,335

(22) Filed: Mar. 4, 2024

Related U.S. Application Data
(63) Continuation-in-part of application No. 18/237,911, filed on Aug. 25, 2023.
(63) Continuation-in-part of application No. 17/175,511, filed on Feb. 12, 2021, now abandoned.

(51) Int. Cl. B23K 26/50; A61B 18/00; A61B 18/20; B23K 26/03; B23K 26/0622; G02B 17/00

(52) U.S. Cl. CPC B23K 26/50; A61B 18/20; B23K 26/032; B23K 26/0622; A61B 2018/00577; A61B 2018/00625; A61B 2018/2035; A61B 2018/2065; G02B 17/004

(57) ABSTRACT

This non-provisional application is a continuation-in-part application filed under 37 CFR 1.53(b) that claims the benefit of United States 35 USC 120 from non-provisional application with U.S. application Ser. No. 18/237,911 filed on Aug. 25, 2023, and from non-provisional application with Pub. No. US 2022/0258277 A1 filed on Feb. 12, 2021. This invention discloses an apparatus of processing object in optical turbid medium using multibeam interference. This invention creates the apparatus having important usages, such as medical no incision laser surgery with deep treating depth, underwater wireless long-distance communication, small attenuation light energy delivery in optical turbid medium. The disclosed apparatus has excellent performance. For example, the created laser scalpel can treat tissue at depths of more than 5 cm in human body with high 3D precision of about 1 μm. The effective light energy delivery distance including underwater wireless communication distance of more than 1000 m in clear seawater.

### CROSS REFERENCES TO RELATED APPLICATIONS

This non-provisional application is a continuation-in-part application filed under 37 CFR 1.53(b) that claims the benefit of United States 35 USC 120 from non-provisional application with U.S. application Ser. No. 18/237,911 filed on Aug. 25, 2023, by the inventor: Shangqing Liu, and from non-provisional application with Pub. No. US 2022/0258277 A1 filed on Feb. 12, 2021 by the inventor: Joyce Liu. The non-provisional application with application Ser. No. 18/237,911 is a continuation application claims the benefit from non-provisional application with U.S. Pat. No. 11,808,568 B2 patented on Nov. 7, 2023. This non-provisional application incorporates non-provisional application with U.S. application Ser. No. 18/237,911 and non-provisional application with Pub. No. US 2022/0258277 A1 in its entirety.

### STATEMENT REGARDING FEDERALLY SPONSORED RESEARCH

Not Applicable.

### REFERENCE TO APPENDIX

Not Applicable.

### FIELD OF THE INVENTION

The present invention relates to an apparatus for processing object in optical turbid medium, and more particularly, to a laser beam apparatus for processing object in the optical turbid medium having light absorption or/and scattering based on using the inner light layer formed by multibeam interference.

### BACKGROUND OF THE INVENTION

As a new kind of processing apparatus, the laser beam processing apparatuses are used to denature, vaporize, ablate, etch, weld, drill, and cut various materials with powerful light energy through photo-chemical, photo-ablative, photo-thermal and photo-mechanical effects. The laser beam processing apparatuses have many advantages: including high power density of larger than 100 MW on an area of smaller than 1 mm²; strong ability to treat almost any materials, especially for very hard or very brittle ones; high precision of up to 1 micrometer; fast processing speed of shorter than Ins; and less cost for operation and apparatus maintenance to treat the materials compared with using other tools. Therefore, such apparatuses have become important and indispensable equipment in many fields of modern life.

A special advantage of the laser beam processing apparatuses is that the processing can be done without damaging the material surface. For example, the laser beam can selectively etch inner area in a bulk photosensitive glass by focusing the laser beam into the glass. Also, in some laser treatments, the laser beam can penetrate human skin layer to heat some underneath tissues which absorb light more effectively, such as the tissue containing chromophore, which can induce necrosis of that tissue for treating disease.

This special ability of processing material without damaging material surface, and furthermore, without damaging deeper inner areas in the beam propagation path before the targeted object, is very useful and even indispensable for some applications. It is expected that this special processing ability can be used for more applications and with deeper processing depth.

However, to reach this goal is very difficult. The reason is that most materials, as the optical media, have strong light absorption and scattering, which not only damage inner materials in the light propagation path and attenuate light energy for processing the targeted object, but also produce scattered light to degrade processing precision. In addition, in some applications, the laser beam processing needs imaging the targeted object for guidance, the scattered light will flood the imaging signal light too. These difficulties make laser beam processing can only be used for the inner object in transparent materials like glass, or for inner object with very shallow depth like the tissue under skin layer with depth of less than 3 mm in photodynamic therapy [see reference: T. J. Dougherty, et al, “Photodynamic therapy (Review),” Journal of the National Cancer Institute, Vol. 90, No. 12, 1998, pp. 889-905].

The apparatus, which can reduce light absorption and scattering and deliver the light energy through longer distance in optical medium and especially in the turbid optical medium with strong light absorption and scattering are extremely valuable and indispensable for some applications. For example, human tissues, water and atmosphere all absorb and scatter light, although their absorption and scattering rates vary large in scale. In the human tissues, as described above, after traveling several millimeters, the light intensity will drop extremely large. In the water, even in the clear seawater, the green light (λ=550 nm), which has best transmittance in the water, can only travel as far as 25 m [W. Hou, “Active Underwater Imaging,” Chapter 4, Ocean Sensing and Monitoring: Optics and Other Methods, SPIE Press Book, 2013, pp. 87-93], which becomes the main difficulty for many underwater applications including underwater light detection and communication. In the atmosphere, if the propagation distance is long, the laser energy will attenuate heavily too, which also produces the difficulty for many applications, including long distance atmosphere light communication, long distance light energy delivery, and so on. In the medical field, especially, if the laser beam can go deeper into the human body without injuring the tissues in the beam propagation path, many diseases can be treated by laser scalpel with high precision, no incision, no bleeding, anti-infection, fast procedure, and moderate cost.

Therefore, the apparatus which can reduce light absorption and scattering and increase the delivery distance of the light energy in the light absorption and scattering materials are widely and crucially needed in modern society.

### PRINCIPLE OF THE INVENTION

An earlier parent nonprovisional application with application Ser. No. 18/237,911 and titled “Apparatus of Inner Light Layer Illumination by Multi-beam Interference for Imaging in Turbid Media”, which is invented by Shangqing Liu and filed on Aug. 25, 2023, disclosed the apparatus which uses multi-beam interference to reduce light absorption or/and scattering and so can increase optical imaging distance significantly in the optical turbid media.

The non-provisional application with application Ser. No. 18/237,911 is a continuation application based on its parent nonprovisional application with U.S. Pat. No. 11,808,568 B2 and titled “Method of Inner Light layer Illumination by Multi-beam Interference and Apparatuses for Imaging in Turbid Media”, which is invented by Shangqing Liu and patented on Nov. 7, 2023, disclosed the method which uses multi-beam interference to reduce light absorption or/and scattering and so can increase optical imaging distance significantly in the optical turbid media.

Another earlier non-provisional application with Pub. No. US 2022/0258277 A1 and titled “Laser Beam Processing Apparatuses and Correspondent Method using Multi-beam Interference”, which is invented by Joyce Liu and filed on Feb. 12, 2021, disclosed the apparatuses and method using multi-beam interference to reduce light absorption or/and scattering and so can increase laser beam processing distance to object significantly in the optical turbid media. The last “Office Action Summary” to this application was mailed to inventor on Dec. 13, 2023. The required period for replying this “Office Action” is 3 months from the mailing date.

The principle disclosed in earlier parent applications is based on using multibeam interference to create destructive interference in the beam propagation path to reduce the illumination light intensity and so to reduce absorption or/and scattering of the media, and to create constructive interference to produce high composite light intensity which forms an inner light layer to illuminate the object for imaging the object or processing the object in the turbid media.

This invention discloses a new substantial improved apparatus of laser beam processing for various objects in the turbid media. In other words, by combining the ideas described in above said earlier parent applications and by improving apparatus structure with substantial changes to create a new laser beam processing apparatus for processing various objects in the turbid media.

This application is a continuation-in-part application of the application with the application Ser. No. 18/237,911 filed on Aug. 25, 2023. This application discloses a new different use, that is, the laser beam processing, based on using the similar method disclosed in its parent application for optical imaging with the application Ser. No. 18/237,911. Under U.S. patent law, the new use for a purpose that is different from what a patent applicant contemplated, and if the purpose is sufficiently distinct, a new patent can be got for that new use. Therefore, the creative work disclosed in this continuation-in-part application applies for a new patent.

This application is also a continuation-in-part application of the application for laser beam processing with the PUB. No. US 2022/0258277 A1 filed on Feb. 12, 2021. This application discloses the apparatus for laser beam processing too but with substantial improved apparatus structure, that is, the apparatus disclosed in this application uses optical fiber device instead of using optical prism device to produce negative dispersion, a long-cavity optical fiber pulse laser instead of normal optical fiber pulsed laser as the light source, and a different processing distance adjuster consisting of hollow optical fiber or hollow thin tube. As a continuation-in-part application, this application can claim the right of priority of filing date for the new different use, that is, for the laser beam processing from the optical imaging based on using the similar method.

The disclosed laser beam processing apparatus can simultaneously reduce the absorption and scattering of the optical turbid media greatly, resulting in great increase of light processing or light energy delivery distance in such media. The designed apparatus has excellent performances. By using this apparatus, the intensity of the light processing or delivery beam can increase more than 14 orders of magnitude than the normal way. The effective processing or delivery distance will reach, for example, more than 5 cm in the human body or more than 1000 m in the clear seawater.

The invented apparatus is designed based on such a principle: in the light beam propagation paths (not only in the beam propagation path to process the object, but also in the signal light return path for imaging the object), the turbid media produce light absorption and scattering, which attenuate the intensities of the processing beams, and even produce light noises to bury the returned signal light. Therefore, one needs to create an apparatus which can make the light beam disappear in the processing path and so without being absorbed and scattered because light absorption and scattering are directly proportional to the light intensity, and also make the light beam only appear on the object and so only to process the object, or only appear at desired position in the turbid medium for light energy delivery. In addition, if needed, the apparatus can further make the reflected signal light disappear in the return path and so without being absorbed and scattered too, and at last only appear at the observation position for imaging. When the object is observed, it means that the object is aimed by the light beam, by increasing the power of the light beam to a required value, the object can be processed. If the intensity of the light beam in the propagation path is still much lower than the intensity of the light beam at the object location, then no light damage or injury in the beam propagation path. This unusual laser processing procedure can be achieved very proximately by using multibeam interference.

The said apparatus of inner light layer formed by multibeam interference for processing object in optical turbid medium comprising:
multimode pulsed laser, the light pulse shot from said multimode pulsed laser contains 10¹ to 10²⁴ polarized light beams with different frequencies;
negative dispersion device including one comprising negative dispersion optical fiber or optical prisms(s), the negative dispersion generated by said negative dispersion device compensates positive dispersion generated within the light pulse traveling path to said object in said optical turbid medium;
processing distance adjuster including one comprising a pair of triangular components, or a hollow optical fiber, or a hollow thin tube, said processing distance adjuster changes the processing distance to said object in optical turbid medium;
a set of optical components including lenses, mirrors, or and prism(s), or and beam splitter(s), wherein said set of optical components make said light pulse from said multimode pulsed laser go in order through said negative dispersion device, said processing distance adjuster, said optical turbid medium, and form an inner light layer on said object.

The said negative dispersion device is used to broaden the width of light pulse shot from the said pulsed laser. Then the light pulse with broadened width enters the medium containing the object. Since the medium containing the object has positive dispersion (almost all of the natural optical media have positive dispersion), the medium positive dispersion will compress the broadened width of the light pulse in the propagation path in the medium to create a short light pulse again on the object in the medium. It will form an inner light layer to illuminate the object in the medium.

When the object is aimed by the laser beams consisting of a single or a series of light pulses, by increasing the power of the laser beams to a required value, the object can be processed.

In addition, the light pulse will be reflected by the object partially, this reflected light pulse with reduced intensity may be used as the signal light pulse if needed. The signal light pulse may return from the object and go along the incident path reversely. During the return path, the width of the signal light pulse is broadened by positive dispersion of the turbid medium first. Then, the width broadened signal light pulse is compressed by the negative dispersion generated by the negative dispersion device afterwards. Finally, the re-shortened signal light pulse is received by an imaging receiver placed at the observation position for image-guided processing.

The principle of this invention for laser beam processing using multibeam interference is described in more detail underneath.

To select N polarized light beams with different angle frequencies ωⱼ (j=0, 1, 2, . . . , N), which have the same or approximately the same amplitudes and the same or approximately the same polarization states. The angular frequency intervals Δω of any two frequency adjacent beams in these N beams are the same or not the same, here supposing the intervals Δω are the same for simplifying the related analyses and calculations. In addition, at a certain moment t the initial phases ϕⱼ (j=0, 1, 2, . . . , N−1) of these N beams are zero. The output beams from mode-locked laser and if their polarization directions are polarized by a polarizer satisfy these conditions [see the reference: P. W. Smith, “Mode-Locking of Laser,” Proc. IEEE, 58(9), 1342-1355, 1970].

The frequencies of these N light beams are in visible region, or/and in infrared, or/and in ultraviolet region(s).

The light fields of these N beams are superimposed to each other on the propagation path first in the negative dispersion generation device and then in the turbid medium containing the object for processing, which produces multiple beam interference. The negative dispersion device has negative dispersion and the turbid medium has positive dispersion. Thus, because these beams have different frequencies and different phase velocities in the dispersive medium or device, the destructive interference of the multiple beams makes the composite amplitude of N beams very small in the most of the propagation paths, so the composite light intensity of N beams is attenuated in the propagation path. Generally, the larger the number N, the smaller the composite light intensity of the N beams caused by destructive interference.

The phase differences between any two frequency adjacent beams change gradually in the propagation path. Since the angular frequency intervals Δω between any two frequency adjacent beams are the same, and the initial phases ϕⱼ (j=0, 1, 2, . . . , N) of these N beams are zero at a previous moment, the phase difference of every two frequency adjacent beams changes gradually from zero to the negative value in the negative dispersion device first, and then from negative value to zero in the positive dispersion medium next, and at a certain position in the turbid medium, the phase differences of all pairs of two frequency adjacent beams become zero or approximately zero at the same time, resulting in constructive interference of the N beams. That is, the amplitudes of the N beams add to each other coherently, and create composite light intensity maximum. If the number N is large enough and the total spectral width is wide enough, the composite light intensity maximum may become extremely large, and the duration of the composite light intensity maximum may become extremely short. Therefore, a short light pulse, that is, a thin inner light layer is formed in the turbid medium for processing the object.

By making the absolute value of the negative dispersion generated by the negative dispersion device be equal or approximately equal to the absolute value of the positive dispersion generated within the light pulse traveling path reaching the object in the medium, but with the opposite sign, the short light pulse shot from the pulsed laser will re-appear, that is, a thin inner light layer will be formed on the object in the medium.

The said apparatus will have the following properties:
1. In most sections of the propagation path in the medium, the composite intensity of the N light beams is very small. Thus, the N light beams will not damage the medium. In addition, because the absorption and scattering of the turbid medium are directly proportional to the intensity of the incident light beam, the absorption and scattering of the turbid medium are greatly reduced. This will not only greatly reduce the transmission attenuation of the light energy, leading to more light energy to process the object, but also greatly reduce the light noise caused by scattering, thus effectively improving the signal to noise ratio of the object imaging.
2. The object is placed at the position of the composite light intensity maximum. The composite light intensity maximum is usually very large by pulse compression effect. It will be large enough for processing various objects in many different applications. In addition, because the reflected light intensity is proportional to the processing light beam intensity, when the processing light is reflected by the object and becomes the return signal light, the signal light intensity will be increased largely too.
3. The duration of the composite light intensity maximum can be very short, resulting in a very thin inner light layer, which will produce high processing precision. Thus, the laser beam processing has high precision not only on the two-dimensional object plane but also along the longitudinal direction. In addition, thin inner light layer can help to obtain high imaging resolution along the direction of depth of field.
4. The return signal light is still mainly composed of the N incident light beams. Although their amplitudes are significantly or even inconsistently attenuated, and their polarization states are changed or even inconsistently changed, as long as these inconsistencies are not very serious (as in most cases), the return propagation of the signal light from the object may be a completely reverse process of N incident light beams to the object (if the signal light returns along the incident route). Therefore, according to the light ray reversible principle [see the reference: D. S. Goodman, “General principles of Geometric Optics,” Chapter 1, Handbook of Optics, Vol. I, 2ed, Edited by M. Bass, and et al, McGRAW-Hill, New York, 1995, p. 1.10], the multiple beam interference can still occur in the return path, resulting in composite light intensity attenuation in most sections of the return path, and composite light intensity maximum appearing at a specific position. This may greatly reduce the absorption and scattering of the turbid media in the return path, preserve the return signal energy and reduce the light scattering noise again. Usually, the imaging receiver is placed at the position of composite light intensity maximum of the return signal light. Sometimes, the signal light travels along a different route from the incident rout to the imaging receiver. Because the required conditions for multiple beam interference may be satisfied too, the destructive interference will also make absorption and scattering of the turbid media small in the signal propagation path, and the constructive interference will make the signal light be received well too.
5. When the number N of the N beams is large enough, or the total spectral width of N beams is wide enough, the composite light intensity maximum can be large enough to process the object in the medium and the composite light intensity in the propagation path can be small enough to not damage the materials.

Generally, the same beam groups having the same characteristics of the said N beams can be repeatedly used to produce a series of composite light intensity maximums which can increase the total energy to process the object, and to produce a series of signal pulses which can increase the total energy to be received by the imaging receiver.

The said polarized light beams may be plane polarized, or elliptically polarized, or circularly polarized light beams, because the beams of plane polarized, elliptically polarized and circularly polarized all can produce interference. The said polarization states include polarization directions of the plane polarized light beams, ellipticity of the elliptically polarized light beams. The said N polarized light beams may be plane, or cylindrical or spherical light beams located in the medium, the thickness of the layer is much thinner than the processing or delivery distance in the medium, or this layer may be further focused to be point or line located in the turbid medium.

Since the plane light beams are used mainly for most applications, the underneath physical analyses and mathematical calculations are based on using plane light beams. For applications of using cylindrical or spherical light beams, the physical analyses and mathematical calculations can follow the similar processes.

When processing the object in the medium, the power of the processing short light pulse is increased to a required value so that the peak intensity of the formed processing short light pulse is high enough for processing the object. Meanwhile, the light intensity of the processing light pulse in the propagation path is kept to be low enough for not damaging the medium material by light pulse broadening.

### BRIEF DESCRIPTION OF THE DRAWINGS

The preferred embodiments of the invented apparatuses will be described underneath. Obviously, these embodiments are not the all apparatuses which can be designed based on the principle of this invention. Basing on the principle of this invention and using existing technical knowledge, the said apparatus embodiments may be modified and alternated. Therefore, the applicants of this invention reserve the rights of all modifications, alternatives, and equivalent arrangements of the invented apparatus embodiments described underneath.

The aforementioned aspects and advantages of the invention will be appreciated from the following descriptions of preferred embodiments and accompanying drawings wherein:

FIG. 1 illustrates a short light pulse is broadened by positive dispersion medium only (in the top), and is broadened by the negative dispersion device first and then is compressed by the positive dispersion medium next (in the bottom).

FIG. 2 illustrates the schematic diagram of optical structure of the medical image-guided no incision laser surgery apparatus.

FIG. 3 illustrates the schematic diagram of optical structure of the underwater image-guided laser energy delivery apparatus.

FIG. 4 illustrates the change of composite light intensity of multibeam interference in dispersive medium. The light intensity I changes with the parameter K. Not that just taking N=20.

### DETAILED DESCRIPTION OF THE INVENTED APPARATUSES

The invented apparatus of laser beam processing for light absorption or/and scattering media has been designed based on the principle described above.

The apparatus comprising: multimode pulsed laser, the light pulse shot from said multimode pulsed laser contains 10¹ to 10²⁴ polarized light beams with different frequencies; negative dispersion device including one comprising negative dispersion optical fiber or optical prisms(s), the negative dispersion generated by said negative dispersion device compensates positive dispersion generated within the light pulse traveling path to said object in said optical turbid medium; processing distance adjuster including one comprising a pair of triangular components, or a hollow optical fiber, or a hollow thin tube, said processing distance adjuster changes the processing distance to said object in optical turbid medium; a set of optical components including lenses, mirrors, or and prism(s), or and beam splitter(s), wherein said set of optical components make said light pulse from said multimode pulsed laser go in order through said negative dispersion device, said processing distance adjuster, said optical turbid medium, and form an inner light layer on said object.

The short light pulse comes from the multimode pulsed laser including mode-locked laser. The same or approximately the same amplitudes of the multiple polarized light beams with beam number N is obtained by using dye to make dispersion compensation to laser cavity gain.

Then, the light pulse enters the input surface of a dispersive medium at t=0. Since the pulse contains N frequency components (the number N is from 10¹ to 10²⁴ or more), that is, N light beams, and also since the light pulse is shot from a mode-locked laser, the phase difference of any pair of two beams corresponding to the angular frequencies ω₁ and ωⱼ₋₁ (j=0, 1, 2, 3, . . . , N−1) in these N beams is zero when the pulse enters the input surface of the medium. Supposing the input surface of the medium is located at the position of x=0, thus, the initial phases φⱼ are zero at x=0 and t=0. The input surface is perpendicular to the x direction.

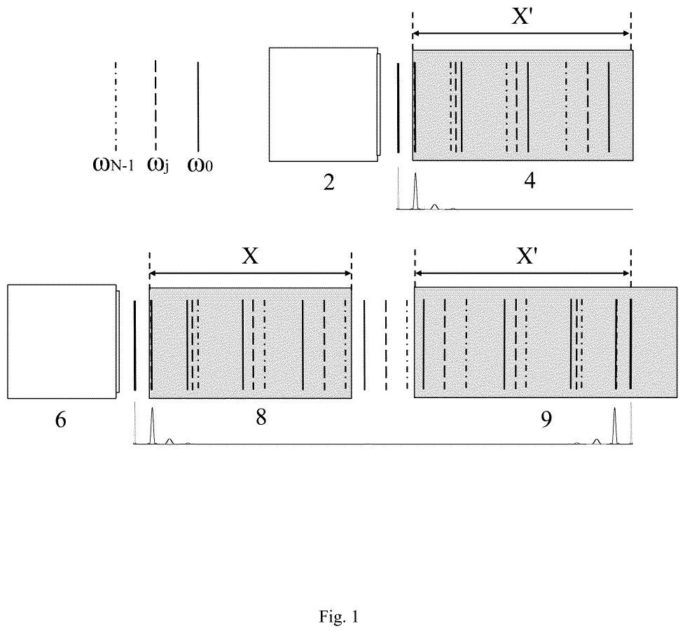

In the most situations, the optical media are positive dispersive media including human body and seawater. When the light pulse enters the positive dispersive medium, the different beams constituting the light pulse travel at different speeds. The higher the beam frequency is, the lower the beam travels. Thus, the pulse broadens and become a beam group with weaker and weaker composite light intensity since destructive interference of the multiple beams as shown in the top of FIG. 1.

In FIG. 1, the light beams are from a mode-locked laser 2. The solid line, dashed line and dotted line represent three planar wavefronts of the beams corresponding to the angular frequencies coo, ωⱼ and ω_{N-1}. If these three wavefronts travel in the positive dispersive turbid medium 4, since ω₀<ωⱼ<ω_{N-1}, the wavefront represented by the solid line travels fastest.

When these three wavefronts travel in a negative dispersion generation device 8 (see the bottom of FIG. 1), their traveling speeds are reversed, that is, the higher the beam frequency is, the faster the beam travels. The light beams are from mode-locked laser 6. If the light pulse enters the device 8 at t=0, three wavefronts are overlapped at the position x=0. In the device 8, since ω₀<ωⱼ<ω_{N-1}, the wavefront presented by the dotted line travels fastest.

Thus, the phase difference Δϕⱼ=ϕⱼ−ϕⱼ₋₁ will change with x from zero to negative value although ωⱼ>ωⱼ₋₁. The destructive interference will occur and grow too in the device 8 with change of Δϕⱼ from zero to negative value.

Note that the shorter the light pulse duration is, the faster the pulse broadens, and the quicker the pulse peak light intensity decreases. It is because the shorter pulse has wider frequency range and contains more frequency components. If defining the decrease time length of the pulse peak intensity from 100% to a significantly small percentage, such as 1%, as the initial broadening period T_{ib}, outside the initial broadening distance D_{ib}=VₐT_{ib}, the light absorption and scattering will become significantly small because the light peak intensity has dropped significantly, where Vₐ is the average speed of the N beams in the dispersive medium. The required initial broadening period T_{ib} or the initial broadening distance D_{ib} depends on the absorption and scattering coefficients of the medium. For the turbid medium with larger absorption or/and scattering, the required T_{ib} or D_{ib} should be shorter. For example, the D_{ib} value should be of the scale of millimeters for medical processing, and be of the scale of the meters for underwater processing. In the same way, the last shortening period Tₗₛ is defined, which is the increase time length of the pulse peak intensity from a very small percentage, such 1% of its maximum value, to the 100% of its maximum value. Because the pulse shortening is the reverse process of the pulse broadening completely, T_{ib} should be equal to Tₗₛ for the same optical dispersive medium.

After leaving the negative dispersion device 8, the broadened light pulse enters the positive dispersive turbid medium 9. The optical path difference of any pair of two frequency adjacent beams of the pulse decreases gradually in the turbid medium 9. The optical path difference between any two of the three wavefronts shown in FIG. 1 also decreases gradually. Thus, in other words, the light pulse broadening terminates, and the light pulse shortening begins.

Let the phase difference between two beams corresponding to angular frequencies ωⱼ and ωⱼ₋₁ in the turbid medium be Δϕⱼ′, the propagation distance of the beam corresponding to angular frequency ωⱼ be xⱼ′ in the turbid medium, and the refractive indexes of the turbid medium corresponding to the angular frequencies ωⱼ and ωⱼ₋₁ be nⱼ′ and nⱼ₋₁′. Then, if the material used in the device for generating the negative dispersion is the same material as the turbid medium, or it has the same or very approximate dispersion property as the turbid medium, then, nⱼ′=nⱼ and nⱼ₋₁′=nⱼ₋₁. Under this condition, if taking xⱼ′=xⱼ, because Δϕⱼ is produced by negative dispersion generation device, we get Δϕⱼ′=−Δϕⱼ. Thus, because Δϕⱼ′−Δϕⱼ=0 for every pair of two frequency adjacent beams, the broadened light pulse will be compressed completely. A thin inner light layer will be formed in the turbid medium 9. Therefore, by making mirrored negative dispersion compensation, the expected thin inner light layer can be formed in the turbid medium [please see more detailed descriptions in earlier parent application with U.S. Pat. No. 11,808,568 B2].

The said light absorption or/and scattering media include human body, animal body, seawater, river water, lake water, pond water, fog, smog, snow, ice, cloud, atmosphere and any gaseous, liquid or solid materials which have light absorption or/and scattering, especially have strong light absorption or/and scattering.

The said laser beam processing includes medical laser beam surgery or treatments, light communications in atmosphere or water, and various light energy deliveries in bulk gaseous, bulk liquid and bulk solid optical turbid media for heating, denaturing, ablating, etching, welding, drilling, vaporizing, hitting, cutting, destroying the objects, and so on.

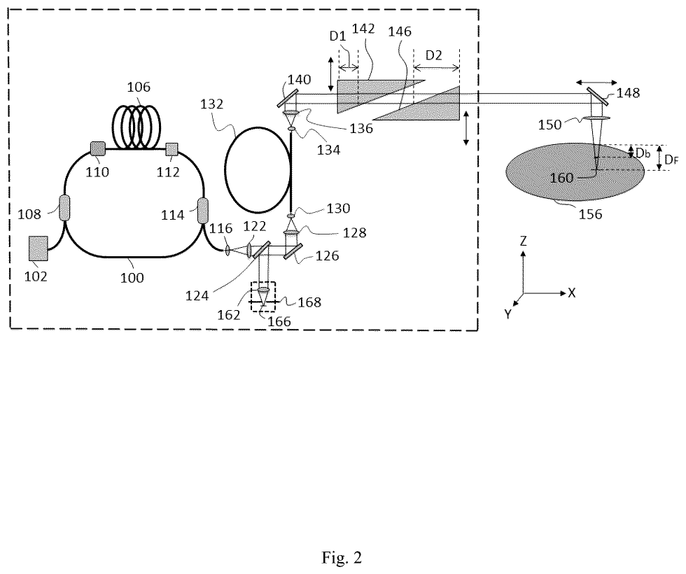

The medical image-guided no incision laser surgery apparatus is described below as the first preferred embodiment of the apparatuses according to the invention. FIG. 2 is the schematic diagram of this apparatus optical structure. Because the sizes of various parts and components differ largely, in order to show necessary details, the shown structure is not drawn in actual proportion.

The short light pulse comes from a mode-locked fiber laser 100, which is pumped by a light-emitting diode 102. The pump light enters a doped fiber 106 through a coupling element 108. When the total spectral bandwidth of the laser output beams needs to be wide, several light-emitting diodes with different emitting frequencies may be used jointly to pump the fiber 106. An optical isolator 110 and a polarization controller 112 are used to ensure unidirectional beam oscillation. The mode-locked fiber laser with multi-wavelength output has been developed maturely [see the references: N. Li, et al, “Cavity-length optimization for high energy pulse generation in a long cavity passively mode-locked all-fiber ring laser,” Applied Optics, 51, 17, 2012, pp. 3726-3730].

The laser beams go out through an optical coupler 114, and then the beam diameters are enlarged by lens 116 and 122. After passing through the beam splitter 124, 10% of the light energy is reflected by the mirror 126 and enters two lenses 128 and 130 for beam diameters shrinking. To use a beam splitter 124 with low transmittance is for less signal light energy loss when the signal light is reflected by beam splitter 124 later. Then, the laser beams enter a negative dispersion optical fiber 132.

In all of earlier parent applications, which are with the U.S. application Ser. No. 18/237,911 filed on Aug. 25, 2023, the Pub. No. US 2022/0258277 A1 filed on Feb. 12, 2021, and the U.S. Pat. No. 11,808,568 B2 filed on Feb. 5, 2021, the negative dispersion devices are designed by using two prisms and two lenses. The negative dispersion devices consisting of prisms have relatively simple structure and have been used for a long time.

However, there is a special requirement for the invented laser beam processing or/and imaging apparatus, which is that such apparatus must produce an ideal negative dispersion to compensate the positive dispersion generated in its corresponding turbid medium. It is a difficult task because that the negative dispersion device comprising the traditional optical prisms can only produce the negative dispersion with sine shaped change rate with the light beam frequency. Therefore, in the earlier parent applications, the negative dispersion device consisting of particular prisms was designed. In such negative dispersion device, the shape of output surface of one prism is manufactured to be non-planar and retroreflective micro-prism mirrors are used to cover this surface. In this way, the produced negative dispersion can have the change rate which is matching the positive dispersion change rate of the correspondent turbid medium [see more detailed descriptions in earlier parent applications]. However, such design of the negative dispersion device increases the manufacture difficulty and reduces dispersion compensation accuracy.

With technology progress, the negative dispersion fibers are able to produce negative dispersions with different change rates (slopes) including without sine change shape. Nowadays, there are various micro-structures built in the optical fibers for producing negative dispersion, such as air-core Bragg grating, square-lattice, dual-core, using modal interactions, and etc. [see the references: G. Ouyang, Y. Xu, and A. Yariv, “Theoretical study on dispersion compensation in air-core Bragg fibers,” Opt. Express 10, 899, 2002; A. H. Bouk, A. Cucinotta, F. Poli and S. Selleri, “Dispersion properties of square-lattice photonic crystal fibers,” Opt. Express 12, 941, 2004; G. Prabhakar, A. Peer, V. Rastogi, and A. Kumar, “Large effective-area dispersion-compensating fiber design based on dual-core microstructure,” Appl. Opt. 52, 4505, 2013; and T. D. Engeness, M. Ibanescu, S. G. Johnson, O. Weisberg, M. Skorobogatiy, S. Jacobs, and Y. Fink, “Dispersion tailoring and compensation by modal interactions in Omni Guide fibers,” Opt. Express 11, 1175, 2002]. Therefore, the optical fiber gives more and better ways to produce ideal (mirrored) negative dispersion to match corresponding turbid medium. In addition, optical fiber negative dispersion devices have less components, small volume and light weight.

Basing on the principles of producing negative dispersion in fiber devices and utilizing existing technical knowledge, the existing negative dispersion fiber devices may be modified and alternated to produce ideal mirrored negative dispersion to match a selected turbid medium. Therefore, the applicants of this invention reserve the rights of all modifications, alternatives, and equivalent arrangements for producing mirrored negative dispersion based on the existing negative dispersion fiber devices and their principles.

For compensating the positive dispersion generated in the turbid medium ideally, all optical path differences produced by all pairs of two frequency adjacent beams in the turbid medium must be generated in the negative dispersion fiber 132 equally but with the opposite signs, that is, a mirrored negative dispersion must be generated.

When the beams travel a distance D in the negative dispersion fiber 132, two beams corresponding to the angular frequencies ωⱼ and ωⱼ₋₁ will produce an optical path difference ΔPⱼ as

where

$$Δ {P}_{j} = D Δ {n}_{j} , j = 0 , 1 , 2 , 3 , … , N - 1 , \qquad ( 1 )$$

$$Δ {n}_{j} = {n}_{j - 1} - {n}_{j} , \qquad ( 2 )$$

and nⱼ and nⱼ₋₁ are the equivalent refractive indexes of the negative dispersion fiber corresponding to the angular frequencies ωⱼ and ωⱼ₋₁. Here, the equivalent refractive index is defined as the refractive index of a man-made optical material (such as a specially designed negative dispersion fiber which has dispersion with a negative change rate).

Suppose the optical path difference produced by two beams corresponding to the angular frequencies ωⱼ and ωⱼ₋₁ in the turbid medium is

where

$$Δ {P}_{j}^{′} = {D}^{′} Δ {n}_{j}^{′} , j = 0 , 1 , 2 , 3 , … , N - 1 , \qquad ( 3 )$$

$$Δ {n}_{j}^{′} = {n}_{j}^{′} - {n}_{j - 1}^{′} , \qquad ( 4 )$$

and nⱼ′ and nⱼ₋₁′ are the refractive indexes of the turbid medium corresponding to the angular frequencies ωⱼ and ωⱼ₋₁. D′ is the traveling distance of the beams in the turbid medium (D′ may be regarded as the processing distance in the turbid medium).

In order to generate mirrored negative dispersion, the negative dispersion device needs to generate the following optical path differences

$$Δ {P}_{j} = - Δ {P}_{j}^{′} = D Δ {n}_{j} = - {D}^{′} Δ {n}_{j}^{′} , \qquad ( 5 )$$

$$j = 0 , 1 , 2 , 3 , … , N - 1 ,$$

Eq. (5) gives the negative optical path difference ΔPⱼ required for two beams corresponding to the angular frequencies ωⱼ and ωⱼ₋₁ in the negative dispersion fiber 132.

The design of the negative dispersion fiber may start with measuring the turbid medium refractive indexes nⱼ′ corresponding to different frequencies within the required range first. Because the number of the frequencies is large, only partial and discrete data need to be measured. Then, one can use a computer to fit refractive index change curve with frequency from the obtained data. There are several dispersion equations for fitting the refractive index change curves, such as Cauchy, Hartmann, Conrady and Kettler-Drude equations, etc. [see the reference: W. J. Smith, “Optical Materials and Interference Coatings,” in Modern Optical Engineering, McGRAW-Hill, 2000, Chapter 7, p. 176].

Then, nⱼ′, nⱼ₋₁′ and Δnⱼ′ can be obtained from the fitted refractive index curve of the turbid medium. And then, according to required processing distance D′, the required negative dispersion change rate of the negative dispersion fiber, that is, the required equivalent refractive indexes nⱼ and nⱼ₋₁ corresponding to the angular frequencies ωⱼ and ωⱼ₋₁, and Δnⱼ may be calculated, which also depend on the chosen value of the lengths D of the negative dispersion fiber by the Eq. (5). Because the value D is variable, which gives more convenience for designing nⱼ, nⱼ₋₁ and Δnⱼ corresponding to the angular frequencies ωⱼ and ωⱼ₋₁ depending on the Eq. (5).

Except the fiber 132, other optical components used in the apparatus will produce positive dispersions too. If the total value of the traveling path lengths of the beams in these components is much less than D′, these additional positive dispersions can be ignored. Otherwise, they need to be compensated too. For simplifying the manufacturing of the apparatus, and also for simplifying physical analysis and mathematical calculation here, all of the optical components in the apparatus had better be made of the material having the same or approximately the same dispersion property as that of the corresponding turbid medium containing the object. To satisfy such a requirement has become relatively easy in recent years. For example, to find a material whose optical property is approximate to the human tissues is not difficult because of the development of tissue simulating phantoms [see the reference: B. W. Pogue, and M. S. Patterson, “Review of tissue simulating phantoms for optical spectroscopy, imaging and dosimetry,” J. Biomed. Opt., 11(4), 02.1-02.16, 2006]. In the optical spectroscopy, imaging, and therapy research fields, such simulating materials have been widely used. The dispersion, absorption and scattering properties of these materials are characteristic of human tissues. Of course, if choosing a material to make the core of fiber 132 (including to fill it into the hollow optical fiber or hollow thin tuber for becoming the processing distance adjuster), its light absorption and scattering should be small for saving light energy. Here, it should be indicated that the negative change rate of the dispersion can be created by micro-structures built in the fiber). If the chosen material is soft, the components, such as prim(s) and beam splitter(s) with relatively large sizes, may be made to be a transparent container and filled with the chosen material.

The diameters of the N light beams from the fiber 132 are expanded by lenses 134 and 136. After reflected by the mirror 140, these beams become parallel beams and enter the processing distance adjuster consisting of two triangular components 142 and 146. Because the length and dispersion change rate of the negative dispersion fiber 132 are fixed, which determines a fixed processing distance D′ in the turbid medium by the Eq. (5), and so every apparatus has a fixed processing distance D′.

Therefore, if the expected processing depth in the human body is D_{b}, the distance changes by adjusting two components 142 and 146 are D₃ and D₄, then to make

$${D}^{′} = {D}_{3} + {D}_{4} + {D}_{b} . \qquad ( 6 )$$

The expected processing depth D_{b} can be adjusted by changing D₃+D₄. Note that the two components 142 and 146 should be made from the same material as the turbid medium or have the same or approximately the same dispersive property as the turbid medium too. Because two triangular components have symmetrical shapes, no unwanted dispersions will be produced by this distance adjuster.

Then, the N parallel beams enter the human body 156 by reflection of the mirror 148. If there is no lens 150, an inner light layer will be created at the depth of D_{b}. The layer thickness is determined by two factors. One is the number N of the beams and the frequency interval Δν(2πΔν=Δω) of the N beams according to the relation of NΔνΔτ=1 [see the reference: W. H. Carters, “Coherence theory,” in Handbook of Optics, McGRAW-Hill, 1995, Vol. I, p. 4.3], where Δτ is the duration of the light pulse when Δϕⱼ=0, which determines the inner light layer thickness δH by δH=VₕΔτ, where Vₕ is the average speed of the N beams in the human body. If the total spectral width of the N beams is wide enough, the layer thickness can be very thin, such as less than 1 μm. Another factor is the initial broadening period T_{ib}. The pulse broadening due to the chromatic dispersion can be estimated as [see the reference: C.-A. Bunge, M. Beckers, and B. Lustermann, Polymer Optical Fibres, Fibre Types, Materials, Fabrication, Characterization and Applications, Elsevier Ltd, Woodhead Publishing, 2017, pp. 47-118]

$$Δ {T}^{′} = {L}^{′} Δ λ {d}_{c} , \qquad ( 7 )$$

where Δλ is the pulse spectral width in wavelength, d_{c} is chromatic dispersion coefficient, and L′ is the propagation distance of the pulse in the dispersive medium, ΔT′ is full width of half maximum (FWHM) of the pulse. For seawater, typical d_{c} values are from 60 ps/nm·km to 300 ps/nm·km in visible region [see the reference: “Seawater intrusion and mixing in estuaries,” Marine Species Introduced Traits Wiki, 2020, marinespecies.org/introduced/wiki/Seawater intrusion and mixing in estuaries]. Because the dispersion coefficients of the human tissues have not been found now, and considering that about 60% of human body is water by weight, thus the dispersion coefficients of the seawater are used to simulate the broadening effect of light pulse propagating in the human body temporarily.

Supposing a 1 fs light pulse with central wavelength of 550 nm enters the human body, thus Δλ=318 nm. Then by taking d_{c}=100 ps/nm·km and L′=1 mm, from the Eq. (7), we get ΔT′=32 fs. It means that a 1 fs pulse is broadened to 32 fs after traveling a short distance of 1 mm, which also means that a pulse of 0.226 μm long is expanded to a pulse of 7.2 μm long after traveling a distance of 1 mm in human body. Because the 1 fs pulse is broadened to 32 fs, peak light intensity of the pulse will drop to below 3.1% of its maximum value. Therefore, since the light pulse of fs level can be broadened fast enough in the human body, if the pulse of fs level is broadened by the negative dispersion in the negative dispersion fiber first, then this broadened light pulse will be shortened fast enough too by the positive dispersion in the human body during the last shortening period Tₗₛ. Thus, the light energy loss due to light absorption or/and scattering during the last shortening period Tₗₛ is small. Fortunately, obtaining ultrafast and high power fs lasers is not difficult nowadays.

In FIG. 2, the N beams are focused by lens 150 when they enter the human body 156. It is for confocal imaging to improve the imaging longitudinal resolution, which will be explained later.

The signal light beams reflected by the targeted tissue return along the incident path reversely. Generally speaking, the N beams constituting the incident pulse will all be reflected by the target tissue. The reflections occur on the interface on the targeted tissue surface and between the two areas with different refractive indexes. The reflectivities of the interface for N beams are not different much usually. During the return path, the signal light pulse will broaden by positive dispersive tissues again as the signal light pulse still contains multiple frequency components, that is, the multiple beams, which results in decrease of the composite intensity of signal light beams, and so results in decrease of the light absorption and scattering in the body again. Then, the signal light beams exit the body. The optical path differences of the signal light beams are further enlarged by positive dispersive processing distance adjuster. After reflected by the mirror 140, the signal light beams enter the negative dispersion fiber 132 again. This time, the broadened signal pulse will be compressed by negative dispersion in the fiber 132. Because the return process of the signal pulse is a completely reverse process of the laser pulse processing process, the detailed analysis and calculation are not repeated.

When the signal beams reach the beam splitter 124 again, the signal light beams travel a distance which equals D′ exactly. Thus, the expected signal pulse appears by constructive interference of the N signal beams. After reflected by beam splitter 124, as beam splitter 124 has high reflectivity, most (90%) of the energy of the signal light pulse is focused on the image plane 166 by lens 162.

If the lens 150 is not used, the designed apparatus has the most popular imaging structure, which can make one point on the object plane become one point on the image plane directly. This structure can easily combine existing ultra-resolution technologies [see the reference: G. Huszka, and M. A. M. Gijs, “Super-resolution optical imaging: A comparison,” Micro and Nano Eng. 2, 7-28, 2019], such as to place a phase filter before the focusing lens 162. In this way, the imaging resolution along the object plane can exceed the theoretical diffraction limit, which is significantly less than the beam wavelengths.

Using the lens 150 is for improving the longitudinal resolution. Because during the last shortening period Tₗₛ in the human body, the light pulse intensity will be significantly large. For example, as described above, within the range of 1 mm, the intensity of a 1 fs pulse drops to 3.1% of its maximum value in the human body. As the initial broadening period and the last shortening period has equal length in the same medium, the effective thickness of a 1 fs pulse will be much larger than its theoretical thickness of approximate 0.226 μm in the human body, which will reduce the longitudinal resolution of the imaging. The confocal imaging can solve this problem [see the reference: S. Inoue, and R. Oldenbourg, “Microscopes,” in Handbook of Optics, McGRAW-Hill, 1995, Vol. II]. By using lens 150 to focus the processing light beams to scan the targeted tissue, and using a spatial pinhole 168 placed before the image plane 166 to block out-of-focus light in image formation, the imaging longitudinal resolution can be increased to wavelength level, that is, less than 1 μm, and with better contrast.

When using the lens 150, the focal point of the lens 150 is at the position with the depth of D_{F} in the human body. It should be indicated here, the formed inner light layer and the focal point 160 locate at different positions, which gives a special benefit of more conveniently changing the processing area size and controlling the processing power density, because the area size, and so the power density of the formed inner light layer can be also changed by adjusting the distance difference of D_{F}−D_{b}.

This apparatus creates a thin inner light layer in the human body 156 in the 2D XY plane. The layer area size may also be changed by the distance difference of D_{F}−D_{b}. In contrast to the focal point scan processing, which is used by many existing 3D imaging or medical treating technologies, this 2D processing simplifies the apparatus optical structure and improves the treating speed.

The mirror 148 can move in the X direction. The apparatus or the human body 156 can move in the Y direction. Thus, by adjusting the processing depth D_{b}, the 3D processing can be achieved in the human body 156. The change of the distance difference of D_{F}−D_{b} is by moving the lens 150 in the Z direction.

Many medical surgeries and treatments need imaging the targeted tissue for guidance. This apparatus has excellent imaging ability. The signal light reflected by the target tissue returns along the incident path reversely. The returned signal light is almost entirely consisted of the previous processing N beams with reduced amplitudes and somewhat changed polarizations. In the returning path, because the returned beams have different frequencies and different phase velocities, thus, the destructive interference makes the composite light intensity small.

When the targeted tissue is observed clearly by observer, it also means that the targeted tissue is aimed by the laser beam exactly. Then by raising the output power of the mode-locked multimode laser to the required level for processing the targeted tissue, the targeted tissue can be vaporized, or ablated, or incised by formed inner light layer, which may be called as laser scalpel. When the processing light power is raised for treating the tissue, the light intensity in the beam propagation path can be still under the safe threshold value since multiple beam interference (see further descriptions below). Afterwards, by reducing the output power of the mode-locked multimode laser to previous level, the result of such no incision laser surgery can be checked by imaging the targeted tissue again.

The method of said invention may further combine a variety of existing technologies to produce a variety of laser processing with or without image guidance. As these are existing technologies and knowledge, no further explanation is needed too.

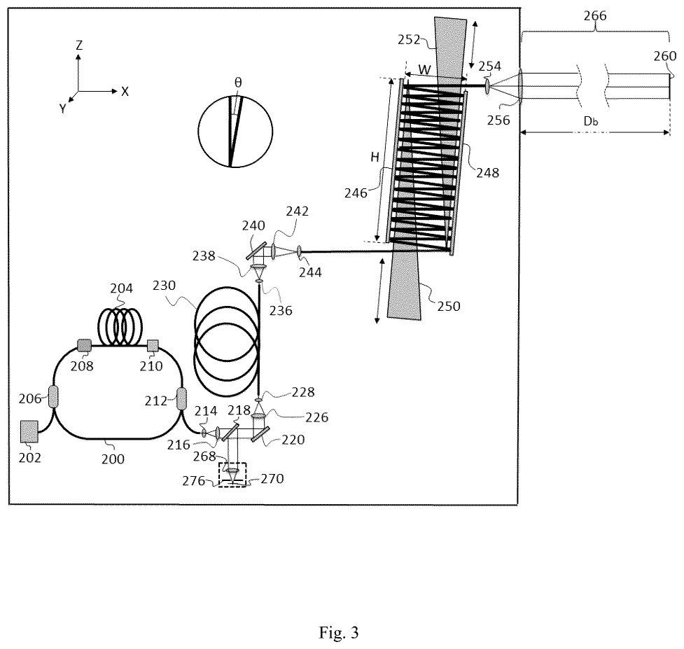

The underwater image-guided laser energy delivery apparatus is described below as the second preferred embodiment of the apparatuses according to the invention. FIG. 3 is the schematic diagram of this apparatus optical structure. Because the sizes of various parts and components differ largely, in order to show necessary details, the shown structure is not drawn in actual proportion too.

In FIG. 3, the mode-locked fiber laser 200 consists of light-emitting diode 202, doped fiber 204, coupling element 206, optical isolator 208, polarization controller 210 and optical coupler 212. The laser beam diameters are enlarged by lens 214 and 216. After passing through beam splitter 218, the laser beams are reflected by mirror 220. Then, the laser beams enter negative dispersion fiber 230 after going through the lenses 226 and 228. In the optical fiber 230, the negative dispersion is produced.

The delivery and imaging distance adjuster is composed of two paralleled mirrors 246 and 248, and two triangular components 250 and 252. Two mirror planes are inclined at an angle θ_{M} to Z axis.

Since the desired delivery and imaging distance underwater is long, after N parallel beams entering the distance adjuster, each beam will be reflected multiple times in the adjuster. Making the diameter of each beam be small, thus each beam can obtain a larger number of reflections between two mirrors. If the expected delivery and imaging distance in the seawater 266 is D_{b}, the distance change by adjusting the components 250 and 252 is ΠD_{w}, and the designed delivery and imaging distance of the apparatus is D′. By making D′=D_{b}+ΠD_{w}, the D_{b} can be adjusted by changing ΠD_{w}. Here, Π is the number of the reflection times of a beam between two mirrors 246 and 248, D_{w} is the travelling distance of a beam in two components 250 and 252 between two reflections. Because the diameters of the beams are small, such as a diameter of 5 mm, the required thickness of the distance adjuster is thin, such as less than 10 mm, so the distance adjuster can have moderate volume and light weight. Furthermore, if required, the distance adjuster can be a composite distance adjuster composed of multiple distance adjusters.

The change of the value of ΠD_{w} is by moving the components 250 and 252 simultaneously along the mirror planes in the opposite directions. Moving the components 250 and 252 simultaneously and in the opposite directions is for offset extra dispersions caused by the triangular shapes of two components.

The delivery and imaging distance adjuster 230 may consist of an optical fiber with hollow core or even a thin plastic or metal tube with hollow core. The material same as the corresponding turbid medium, that is, the turbid medium containing the object, or the material having the same or approximately the same dispersion property same as the corresponding turbid medium is filled into the optical fiber, or the thin tube. By changing the amount of the filled material in fiber or thin tube, that is, by changing the traveling distance of the light pulse in the filled material in the optical fiber or thin tube to adjust the delivery and imaging distance in the turbid medium. The delivery and imaging distance adjuster consisting of the hollow core optical fiber or thin tube has advantages of simple structure, smaller volume and light weight.

After the diameters of the N beams are expanded by the lens 254 and 256, the N parallel beams enter the seawater 266 and form an inner light layer on the object plane 260 at the position with the distance of D_{b}.

This apparatus forms a 2D energy delivery, which will simplify the delivery and imaging optical structure and improve delivery and imaging speed, since a 2D inner light layer is formed in the YZ plane in the seawater 266. The layer area is determined by the cross-sectional area of the N beam group. The Group of the N beams can scan up and down, from the right to the left, in order to achieve large range 3D energy delivery and imaging in the seawater 266.

Compared with the medical processing and imaging apparatus, the depth resolution requirement of the underwater delivery and imaging is generally much lower. In the seawater, the expected delivery and imaging distance is from several meters to even kilometers, thus the depth resolution of 1 mm to 10 mm is very enough generally, which is 3 to 4 orders of magnitude lower than the requirement for medical processing and imaging apparatus. Therefore, the total spectral bandwidth of the N beams is 3 to 4 orders of magnitude narrower than that of the medical processing apparatus too.

The signal light produced by reflection from the object in the formed light layer returns back along the incident path reversely, and going through a process similar to that in the medical processing and imaging apparatus described above. At last, the signal light beams are reflected by the beam splitter 218 and create the expected signal light pulse, which is focused on the image plane 270 by lens 268.

In the same way, in order to improve the longitudinal resolution of imaging, a spatial pinhole 276 is placed before the image plane 270 to form the confocal imaging.

The lens 268 and the 2D image plane 270 also form the most common camera structure, which makes one object point become one image point, and so it is easy to get high imaging resolution and fast imaging speed.

The returned signal light rays from the points of the object plane are not drawn in FIG. 3. In addition, the parallel delivered light beams may be focused on the object plane, then by point scanning way to focus the light energy to a tiny point on the object plane and to produce the image on the image plane 270.

When the object in the seawater is observed clearly by observer, it means that the light energy can be delivered to that position rightly by raising or not raising the output power of the mode-locked multimode laser. For example, the underwater light communication may not need to raise the laser beam power. Some other applications, such as to hit shark or other dangerous creatures needs to raise the laser beam power. If the laser beam power is raised to the level high enough, more light energy can be delivered too. Afterwards, by reducing the output power of the mode-locked multimode laser to the imaging level, the result of light energy delivery can be checked by imaging the aimed object.

Similarly, based on the principle of the invention, a variety of existing technologies can be combined to create a variety of new functions for underwater apparatus. Since these works may be done by using the existing knowledge, no more explanations are given here.

### BRIEF DESCRIPTION OF PERFORMANCES OF THE INVENTED APPARATUSES

In the following description, the laser beam processing and imaging, and light energy delivery and imaging performances of the said apparatuses are given.

In order to simplify the description, the performance demonstrations for said apparatuses are based on demonstrating their imaging performances. The extra performances for said apparatus, such as much thinner thickness of the formed inner light layer and much larger difference between the intensity of the formed inner light layer and the intensity of the light beams in the propagating path, can also be demonstrated simultaneously.

Following the way of the analyses and derivations described in the earlier application titled “Method of inner light layer illumination by multi-beam interference and apparatuses for imaging in turbid media” with U.S. Pat. No. 11,808,568 B2, we have the composite light intensity of the multibeam interference of the N beams in dispersive medium, which is

$$I = {A}^{2} ( x ) \frac{{cos}^{2} [ \frac{( N - 1 ) ( K Δ ω )}{2} ] {sin}^{2} ( \frac{NK Δ ω}{2} )}{{sin}^{2} ( \frac{K Δ ω}{2} )} . \qquad ( 8 )$$

where K=t−(2xn₀′/C), A is the amplitude of the N beams (supposing the amplitudes of the N beams are the same), t is the time, x is the beam traveling distance in the turbid medium, n₀′ is the refractive index of the turbid medium corresponding to the angular frequency ω₀, C is light speed in vacuum.

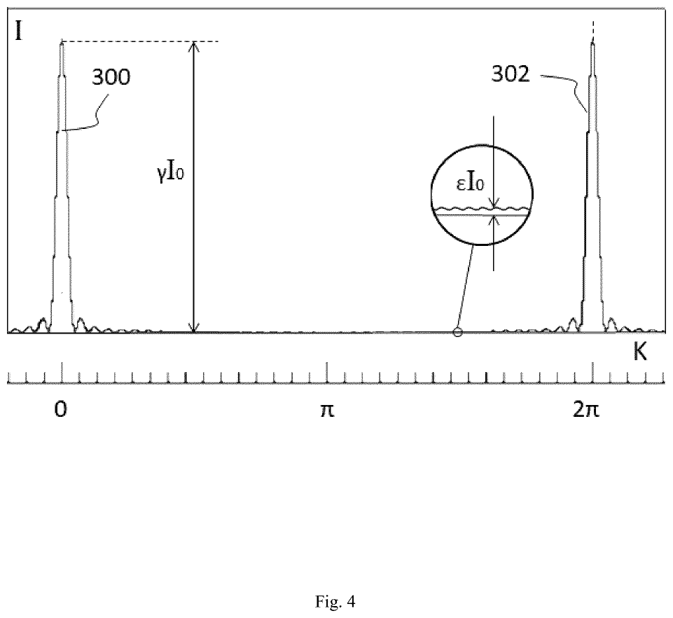

When KΔω becomes zero, the value of I goes to the maximum. The results of numerical calculations by Eq. (8) are shown in FIG. 4 and Table 1. In FIG. 4, I changes with parameter K. 300 and 302 are two composite light intensity maximums. Using K as the unit of transverse coordinate is for avoiding complicated theoretical derivations, and the essential characteristics of the multibeam interference in optical dispersive medium can still be shown. The I curve in FIG. 4 may be regarded as the change of the composite light intensity of N beams with x at the moment when the inner light layer is recreated. In the calculations, taking A=1. In order to show the width of the composite light intensity maximum obviously, just taking N=20.

TABLE 1
Signal Intensity Enhancement at Different Imaging
Depth in Human Body by Multibeam Interference.

| N | γ | ε | Factor | D′ = 2 cm | D′ = 5 cm | D′ = 10 cm | D′ = 15 cm | D′ = 20 cm |
|---|---|---|---|---|---|---|---|---|
| 1.00E+01 | 1.00E+02 | 5.05E−14 | ξ | 1.10E−01 | 1.10E−01 | 1.10E−01 | 1.10E−01 | 1.10E−01 |
| 1.00E+02 | 1.00E+04 | 6.11E−10 | ξ | 1.10E+01 | 1.10E+01 | 1.10E+01 | 1.10E+01 | 1.10E+01 |
| 1.00E+03 | 9.97E+05 | 6.21E−06 | ξ | 1.10E+03 | 1.10E+03 | 1.09E+03 | 1.09E+03 | 1.09E+03 |
| 1.00E+04 | 7.08E+07 | 2.80E−06 | ξ | 7.79E+04 | 7.79E+04 | 7.78E+04 | 7.78E+04 | 7.78E+04 |
| 1.00E+05 | 7.08E+09 | 1.13E−05 | ξ | 7.78E+06 | 7.78E+06 | 7.76E+06 | 7.75E+06 | 7.74E+06 |
| 1.00E+06 | 7.08E+11 | 9.35E−06 | ξ | 7.79E+08 | 7.78E+08 | 7.77E+08 | 7.76E+08 | 7.75E+08 |
| 1.00E+07 | 7.08E+13 | 5.31E−06 | ξ | 7.79E+10 | 7.78E+10 | 7.78E+10 | 7.77E+10 | 7.77E+10 |
| 1.00E+01 | 1.00E+02 | 5.05E−14 | α (dB) | 2.63E+02 | 6.27E+02 | 1.23E+03 | 1.84E+03 | 2.45E+03 |
| 1.00E+02 | 1.00E+04 | 6.11E−10 | α (dB) | 2.83E+02 | 6.47E+02 | 1.25E+03 | 1.86E+03 | 2.47E+03 |
| 1.00E+03 | 9.97E+05 | 6.21E−06 | α (dB) | 3.03E+02 | 6.67E+02 | 1.27E+03 | 1.88E+03 | 2.49E+03 |
| 1.00E+04 | 7.08E+07 | 2.80E−06 | α (dB) | 3.21E+02 | 6.85E+02 | 1.29E+03 | 1.90E+03 | 2.51E+03 |
| 1.00E+05 | 7.08E+09 | 1.13E−05 | α (dB) | 3.41E+02 | 7.05E+02 | 1.31E+03 | 1.92E+03 | 2.53E+03 |
| 1.00E+06 | 7.08E+11 | 9.35E−06 | α (dB) | 3.61E+02 | 7.25E+02 | 1.33E+03 | 1.94E+03 | 2.55E+03 |
| 1.00E+07 | 7.08E+13 | 5.31E−06 | α (dB) | 3.81E+02 | 7.45E+02 | 1.35E+03 | 1.96E+03 | 2.57E+03 |

In the Table 1, N is the number of the beams participating in the interference. γ is the enhancement factor of the composite light intensity maximum γI₀. ε is the attenuation factor of the remaining composite light intensity ϵI₀ between two composite light intensity maximums (see FIG. 4). I₀ is the incident intensity of each beam of the N beams. We see that γ increases with N increase rapidly. The detailed description about the calculations is given in the earlier application with U.S. Pat. No. 11,808,568 B2.

The calculation results shown in FIG. 4 and Table 1 indicate that the larger the N value, the higher the composite light peak intensity γI₀, the narrower the full-width of half maximum of the composite light peak intensity γI₀. At the same time, the larger the N value, the smaller the remaining composite light intensity εI₀ between two peak light intensities.

The numerical calculations show that when the N changes from 10¹ to 10⁷, the enhancement factor γ of the composite light peak intensity γI₀ changes from 10² to 10⁴, and the attenuation factor ε of the remain composite light intensity εI₀ changes from 10⁻¹⁴ to 10⁻⁶. The calculation results are shown in Table 1.

In the Table 1, the difference between the composite peak light intensity γI₀ and the remaining composite light intensity εI₀ may be over 18 orders of magnitude. Such large intensity difference can certainly give plentiful room to avoid light injury for human tissues or light damage for materials in the laser beam propagation path. In the medical surgery applications, the light power density of less than 10 mW/cm2 is safe for human tissues including skin [see reference: T. J. Dougherty, et al, “Photodynamic Therapy (Review),” Journal of the National Cancer Institute, Vol. 90, No. 12, 1998, pp. 889-905]. And the power density of higher than 10 W/cm2 can ablate most targeted tissues in human body without problem. The intensity difference between 10 mW/cm2 and 10 W/cm2 is just 3 orders of magnitude. Therefore, no incision laser surgery can be completed by the invented apparatus.

Now, based on the actual absorption and scattering situations of the human body and seawater, the processing and imaging, and energy delivery and imaging light intensity changes and imaging signal sensitivity enhancements can be calculated.

In the human body, the absorption coefficient and scattering coefficient are different for different tissues. Here, the average absorption coefficient μ_{ba}=0.397 mm-1 and scattering coefficient μ_{bs}=1 mm⁻¹ of the human blood are taken for the whole body temporarily [see above Dr. M. C. Hillman's doctoral thesis]. Although taking the coefficients of blood for whole human body is differ from the actual situation, as mentioned above, since there is a large amount of blood in the human body and the coefficients of the blood have approximate order of magnitude as those of the most human tissues, such treatments can give approximately reasonable results. In addition, there is a practical method for determining the expected processing or imaging distance in the media consisting of the compositions with different refractive indices. it will be given at the last of this invention. The light reflectance R is assumed for the case that light is reflected from the interface between the blood and adipose. The refractive indices of the blood and adipose are taken as 1.3771 and 1.4714, respectively (correspondent to the wavelength of 680 nm). Then, according to the Fresnel formula, at the boundary of two media with different refractive indices of n₁ and n₂, the amplitude reflectance r is

$$r = \frac{{n}_{1} - {n}_{2}}{{n}_{1} + {n}_{2}} , \qquad ( 9 )$$

[see the reference: J. M. Bennett, “Polarization,” Chapter 5, Handbook of Optics, Vol. I, 2ed, Edited by M. Bass, and et al, McGRAW-Hill, New York, 1995, p. 5.7], and the light intensity reflectance R=r². We get the light intensity reflectance R=0.0011 for this interface, Also according to the intensity enhancement factor γ and the intensity attenuation factor ε shown in the Table 1, we get the light signal intensity change factor ξ and the imaging sensitivity enhancement factor α values with different imaging distances of 2 cm, 5 cm, 10 cm, 15 cm and 20 cm in the human body as the follows in Table 1.

The absorption coefficient and scattering coefficient of the seawater vary a lot according to the different situations, here taking the clear seawater as the example. The absorption coefficient μ_{wa} of clear seawater is 0.0196 m⁻¹, and its scattering coefficient μ_{ws} is 0.0212 m⁻¹ [see above reference written by C. D. Mobley]. The refractive index of water at the wavelength of 550 nm is 1.336. Assuming the refractive index of the object is 1.6 (note that the optical glass refractive index range is 1.5 to 2.0), then an assumed light intensity reflectance of R=0.00809 for object in clear seawater is obtained. According to the intensity enhancement factor γ of the composite light peak intensity and the intensity attenuation factor ε of the remain composite light intensity in the Table 2, we get the light signal intensity change factor ξ and the imaging sensitivity enhancement factor α values with different imaging distances of 200 m, 500 m, 1000 m, 1500 m and 2000 m in the clear seawater shown in Table 2.

From Table 1 and 2, we can see that the signal composite light intensity maximum is much higher than the signal intensity of the normal imaging. For example, when D′=5 cm for medical processing and imaging, or D′=500 m for underwater energy delivery and imaging, the intensity enhancement factor ξ is more than 1.1×10³ when N>10³. It means that when N>10³, and the total spectra width of N beams is wide enough, the peak intensity of the imaging signal light pulse can be higher than NI₀. Note that NI₀ is the average value of the total intensity of the N beams (if the N beams are incoherent light beams). Of course, the extreme high pulse peak intensity is always with the extreme narrow pulse duration usually, and so the energy of each pulse may be very low. However, as long as the signal to noise ratio is high, the required signal energy can be got by receiving repeated signal pulses. It can be seen that the value ξ is still high even when D′=20 cm for medical processing and imaging, or D′=2000 m for underwater light delivery and imaging. Therefore, there is good potential to get the light signal intensity enhancement factor of near 2000 dB, whose corresponding processing and imaging depth is 20 cm in the human body, and corresponding energy delivery and imaging distance is 2000 m in the clear seawater. Considering the approximations are made in the calculations, the light signal intensity enhancement factor of 600 dB is taken for representing the apparatus performances, whose corresponding processing and imaging depth is 5 cm in the human body, and whose corresponding energy delivery and imaging distance is 1000 m in the clear seawater.

TABLE 2
Signal Intensity Enhancement at Different Imaging
Distance in Seawater by Multibeam Interference.

| N | Γ | ε | Factor | D′ = 200 m | D′ = 500 m | D′ = 1000 m | D′ = 1500 m | D′ = 2000 m |
|---|---|---|---|---|---|---|---|---|
| 1.00E+01 | 1.00E+02 | 5.05E−14 | ξ | 1.10E−01 | 1.10E−01 | 1.10E−01 | 1.10E−01 | 1.10E−01 |
| 1.00E+02 | 1.00E+04 | 6.11E−10 | ξ | 1.10E+01 | 1.10E+01 | 1.10E+01 | 1.10E+01 | 1.10E+01 |
| 1.00E+03 | 9.97E+05 | 6.21E−06 | ξ | 1.10E+03 | 1.10E+03 | 1.09E+03 | 1.09E+03 | 1.09E+03 |
| 1.00E+04 | 7.08E+07 | 2.80E−06 | ξ | 7.79E+04 | 7.79E+04 | 7.78E+04 | 7.78E+04 | 7.78E+04 |
| 1.00E+05 | 7.08E+09 | 1.13E−05 | ξ | 7.78E+06 | 7.78E+06 | 7.76E+06 | 7.75E+06 | 7.74E+06 |
| 1.00E+06 | 7.08E+11 | 9.35E−06 | ξ | 7.79E+08 | 7.78E+08 | 7.77E+08 | 7.76E+08 | 7.75E+08 |
| 1.00E+07 | 7.08E+13 | 5.31E−06 | ξ | 7.79E+10 | 7.78E+10 | 7.78E+10 | 7.77E+10 | 7.77E+10 |
| 1.00E+01 | 1.00E+02 | 5.05E−14 | α (dB) | 2.63E+02 | 6.27E+02 | 1.23E+03 | 1.84E+03 | 2.45E+03 |
| 1.00E+02 | 1.00E+04 | 6.11E−10 | α (dB) | 2.83E+02 | 6.47E+02 | 1.25E+03 | 1.86E+03 | 2.47E+03 |
| 1.00E+03 | 9.97E+05 | 6.21E−06 | α (dB) | 3.03E+02 | 6.67E+02 | 1.27E+03 | 1.88E+03 | 2.49E+03 |
| 1.00E+04 | 7.08E+07 | 2.80E−06 | α (dB) | 3.21E+02 | 6.85E+02 | 1.29E+03 | 1.90E+03 | 2.51E+03 |
| 1.00E+05 | 7.08E+09 | 1.13E−05 | α (dB) | 3.41E+02 | 7.05E+02 | 1.31E+03 | 1.92E+03 | 2.53E+03 |
| 1.00E+06 | 7.08E+11 | 9.35E−06 | α (dB) | 3.61E+02 | 7.25E+02 | 1.33E+03 | 1.94E+03 | 2.55E+03 |
| 1.00E+07 | 7.08E+13 | 5.31E−06 | α (dB) | 3.81E+02 | 7.45E+02 | 1.35E+03 | 1.96E+03 | 2.57E+03 |

For the underwater energy delivery and imaging, hitting a shark perhaps needs a light power density of about 100 W/mm2 because the light power with density of 500 W/mm2 can cut a steel plate [see reference: Miyamoto and H. Maruo, “Mechanism of laser cutting,” Welding in the World, Le Soudage Dans Le Monde, Vol. 29, No. 9/10, 1991, pp. 283-294] Therefore, if the light power density of the laser beam is 50 mW/mm2 in the propagation path, which should not heat the water obviously. The difference between 50 mW/mm2 and 500 W/mm2 is 4 orders of magnitude. Therefore, good underwater light energy delivery can also be completed by the invented apparatus.

The said apparatuses have such great performances is not strange, because it is created by multibeam interference. In the past, multibeam interference has demonstrated its astonishing abilities, such as to create extremely short light pulse of dozens of attoseconds (1 attosecond=10⁻¹⁸ s) and extremely strong light power of several terawatts (1 terawatt=10¹² watts). They are the fastest-ever and strongest-ever man-made events until now. In the future, the multibeam interference will surely make more technical contributions.

At last, we give the practical method for adjusting the additional distance required by the distance adjuster to get accurate expected laser beam processing and imaging depth in the human body, or light energy delivery and imaging distance underwater. In human body or underwater, there are various compositions with different refractive indices, so it is difficult to find the accurate value of the additional distance determined by all refractive indices of the compositions in the human body or water. However, there is a practical way to overcome this difficulty. Because when the angular phase differences between any frequency adjacent beams become zero, the composite light intensity maximum emerges certainly. Therefore, somewhat like to search for a music station by tuning the frequency of a radio, no matter what the accurate distance of the targeted tissue or object position is, one just needs observing into the human body or water and adjusting the distance adjuster at the same time. When the searched targeted tissue or object appears in the visual field (by camera) and becomes clear, the accurate expected processing and imaging depth, or delivery and imaging distance is achieved.

Similarly, based on the principle of this invented apparatus, a variety of existing technologies can be combined to create a variety of new functions. Since these works may be done by using the existing knowledge, no more descriptions are given here.

### FURTHER EXPLANATION OF THE INVENTION

The apparatuses disclosed in this invention are not obvious modifications or alternatives of the optical imaging apparatuses disclosed in the earlier parent applications with the application Ser. No. 18/237,911 filed on Aug. 25, 2023 and U.S. Pat. No. 11,808,568 B2 filed on Feb. 5, 2021. In other words, the laser beam processing or delivery apparatuses disclosed in this invention are not the obvious modifications by simply increasing the power of the illumination light pulse of the optical imaging apparatuses disclosed in the earlier patent applications. In order to explain it adequately, a detailed explanation is given below.

The invented laser beam processing and delivery apparatuses and the disclosed optical imaging apparatuses are for solving different problems in different technological fields, and so they need different functions and performances for different requirements. The invented laser beam processing and delivery apparatuses aim at different tasks including important no incision laser surgery and underwater long-distance wireless communication. Therefore, the laser beam processing and delivery apparatuses must be designed with substantial changes from the prior arts for having required functions and performances. The substantially different technical requirements are originated from the following three main reasons:

1. There is a significant difference between the light powers required for optical imaging and no incision laser surgery. This power difference is more than many orders of magnitude. For example, the light power density must be less than 10 mW/cm² for optical imaging, otherwise, the human body tissues including skin will be hurt. However, the light power density for laser surgery, such as for ablating most tissues, must be more than 10 W/cm². In addition, to ablate hard bones or to vaporize some tiny tissues, higher light power densities are needed.

However, producing the required much higher light power density for no incision laser surgery can't be accomplished by simply increasing the power of the illumination short light pulse of the imaging apparatus. If the power of the illumination short light pulse is simply increased, although the power density of the formed inner light layer falling on the targeted object can be increased satisfactorily (such as increased by more than 3 orders of magnitude), in the traveling path of the N illumination light beams in front of the formed inner light layer, the (total) light power density of the N illumination beams will be increased with the same proportion (that is, also increased by more than 3 orders of magnitude). Because the required power density for laser surgery is much higher than the required power density for optical imaging, the power density in the light beam traveling path will be increased with big jump to the level which will much over the safety threshold for not hurting the body tissues. Therefore, one must change the apparatus design for laser beam processing substantially. For example, to make the apparatus for no incision laser surgery with such performance: when the power density of the formed inner light layer is increased to required very high level which can process the target tissue satisfactorily, the (total) power density of the N light beams in their traveling path must be still kept being lower than the safety threshold value for not hurting other healthy tissues in front of the target tissue and during the light beam traveling path. This is a much more stringent requirement, which can't be accomplished in the simple way given by optical imaging apparatuses described in prior arts.

2. Another substantially different requirement for the no incision laser surgery application is that the no incision laser surgery must have high surgery precision, which is also a unique advantage for most general laser surgeries. For the no incision laser surgery application in this invention, the designed precision (for longitudinal direction) is about 1 micrometer.

Compared with the precision along the object plane, the precision along the longitudinal direction is more difficult to achieve by optical means. The reason is that the precision along the object plane can be controlled by changing the shape and size of the formed inner light layer along the object plane, which is easier to complete.

However, the longitudinal precision of the laser surgery depends on the thickness of the formed inner light layer. In other words, the 3D precisions of laser surgery depend on the shape, size, and thickness of the formed inner light layer, because the formed inner light layer will interact with the tissue(s) within the area/space of the formed inner light layer directly. Thus, the thickness of the formed inner light layer determines the longitudinal precision of the laser surgery. However, to shorten the thickness of the formed light layer is much more difficult than to change its shape and size by optical means when required layer thickness is very thin.

The thickness of the formed inner light layer for laser surgery must be much thinner than that for optical imaging. It is because that, for optical imaging, the object may be illuminated by an inner light layer with thicker thickness first. Then, all of the 3D precisions/resolutions of optical imaging can be improved greatly by camera, such as using a confocal imaging camera or even adding an ultra-resolution phase filter before the camera.

No descriptions for making the method and its related apparatus meet such extremely stringent technical requirements in prior arts of optical imaging.

3. The third reason is caused by the application of underwater long distance wireless optical communication. The invented method for underwater communication wishes to increase the distance of communication much more than the distance of underwater optical imaging in the disclosed prior art. As described in the specification, during the light beam traveling path, the light beam power decays exponentially with the linear increase of the traveling distance. Thus, when using the way of increasing illumination light pulse power simply, if one wants to increase the communication distance significantly even in the linear manner, one must increase the power of the illumination light pulse significantly and exponentially. However, increasing the power exponentially will reach the output limitation very soon for any attainable laser. For example, increasing the (average) output power from several watts to several kilowatts is just a power rise of 3 orders of magnitudes, however, tripling the distance of underwater optical imaging is far from enough for underwater optical communication. In addition, the several kilowatts are almost the limitations of (average) output power for most common lasers.

Therefore, to make the thickness of the formed inner light layer much thinner than that for optical imaging, to produce the difference of power densities between the formed inner light layer and that in the traveling path much larger than that for optical imaging, and to increase the underwater communication distance much longer than the underwater imaging distance, the apparatuses for laser beam processing and delivery must be changed substantially to satisfy above mentioned extremely stringent requirements.

In the prior arts, the laser used for applications is a general mode-locked pulsed laser, which certainly doesn't satisfy above mentioned stringent requirements. After searching and calculating, an optical fiber laser pumped by several light-emitting diodes has been adopted as the light source for laser beam processing, which can obtain extremely wide spectrum bandwidth.

First, using such an optical fiber laser is for creating an inner light layer with extremely thin thickness because the wider the spectrum bandwidth of the output light pulse, the shorter the width of the output light pulse, and the thinner the thickness of the formed inner light layer. The optical fiber laser is easily pumped by several light-emitting diodes with different frequency radiations, and thus to emit the light pulse with very wide spectrum bandwidth.

Second, the optical fiber laser can get a much longer cavity length than the other type of lasers [see reference paper titled “Cavity-length optimization for high energy pulse generation in a long cavity passively mode-locked all-fiber ring laser” cited in the specification of the earlier parent application with Pub. No. US 2022/0258277 A1 filed on Feb. 12, 2021], which is for producing much more output wavelengths, that is, for produce much more light beams with different angular frequencies to participate in the multibeam interference, which can create the required much larger difference of power densities between the formed inner light layer and that in the traveling path. There is a relationship between frequency interval Δν and laser cavity length L:

$$Δ v = \frac{C}{2 {n}^{′} L} , \qquad ( 10 )$$

where C is speed of light in vacuum, n′ is the refractive index of the medium in the laser cavity [see the reference “Longitudinal laser modes” in Chapter 11.4, Handbook of Optics, Vol. I, McGRAW-Hill, New, York. 1995, p. 11.21]. One can see that, the longer the length L of the laser cavity, the narrower the frequency intervals Δν of N light beams, and the more the number of the frequency components of the output light pulse if the total spectrum range of the output light pulse is fixed. Then, combining with the increased ultra-wide bandwidth of the laser pulse spectrum, much more light beams with different wavelengths can be obtained, which results in a much larger difference of power densities between the formed inner light layer and that in the traveling path by multibeam interference. The inventors of this application want to indicate that although the optical fiber laser was mentioned for being the light source for the optical imaging apparatuses in the prior arts, there was no words of using long cavity optical fiber laser for being the light source for the imaging apparatuses in the prior arts. The optical fiber lasers with normal cavity length and long cavity length are two different technical concepts.

Another essential different of the apparatus structure is using negative dispersion fiber, or hollow optical fiber, or hollow thin tube to replace the negative dispersion device comprising optical prisms, which has less components, smaller volume, and light weight for producing negative dispersion. Furthermore, it gives more and better ways for producing ideal mirrored negative dispersion. This essential improvement of the apparatus structure has not disclosed in all of prior arts.

As emphasized above, the laser beam processing and delivery apparatuses disclosed in this application has a specially designed light source which has ultra-wide spectrum bandwidth with ultra-many frequencies than those offered by optical imaging apparatuses disclosed in the prior arts. In addition, the laser beam processing and delivery apparatuses disclosed in this application has an essential structure improvement by using negative dispersion fiber, or hollow optical fiber, or hollow thin tube, which is also a key different from the prior arts. Therefore, the laser beam processing and delivery apparatuses disclosed in this application have sufficiently distinct purpose from the prior arts.

I claim:

1. An apparatus of inner light layer formed by multibeam interference for processing object in optical turbid medium, the apparatus comprising:
multimode pulsed laser, the light pulse shot from said multimode pulsed laser contains 10¹ to 10²⁴ polarized light beams with different frequencies;
negative dispersion device including one comprising negative dispersion optical fiber or optical prisms(s), the negative dispersion generated by said negative dispersion device compensates positive dispersion generated within the light pulse traveling path to said object in said optical turbid medium;
processing distance adjuster including one comprising a pair of triangular components, or a hollow optical fiber, or a hollow thin tube, said processing distance adjuster changes the processing distance to said object in optical turbid medium;
a set of optical components including lenses, mirrors, or and prism(s), or and beam splitter(s), wherein said set of optical components make said light pulse from said multimode pulsed laser go in order through said negative dispersion device, said processing distance adjuster, said optical turbid medium, and form an inner light layer on said object.

2. The apparatus of claim 1, wherein said optical turbid medium consists of human body, or animal body, or seawater, or river water, or lake water, or pond water, or fog, or smog, or snow, or ice, or cloud, or atmosphere, or and gaseous, or liquid, or solid material having light absorption or/and scattering.

3. The apparatus of claim 1, wherein said processing object consists of medical laser surgery or treatment for human or animal body, or light communication in atmosphere or water, or light energy delivery in gaseous, or liquid, or solid material having light absorption or/and scattering for illuminating, heating, denaturing, ablating, etching, welding, drilling, vaporizing, hitting, cutting, and destroying said object.

4. The apparatus of claim 1, wherein said multimode pulsed laser is mode-locked laser, or multimode wideband pulsed laser with wide spectrum band of larger than 2 nm, or long cavity optical fiber laser of longer than 1 m.

5. The apparatus of claim 1, wherein said different frequencies of said polarized light beams are in visible region, or/and in infrared, or/and in ultraviolet region(s).

6. The apparatus of claim 1, wherein said 10¹ to 10²⁴ polarized light beams are plane polarized, or elliptically polarized, or circularly polarized.

7. The apparatus of claim 1, wherein said 10¹ to 10²⁴ light beams are plane, or cylindrical, or spherical light beams.

8. The apparatus of claim 1, wherein said the negative dispersion generated by said negative dispersion device compensates positive dispersion generated within the light pulse traveling path to said object in said optical turbid medium is to make the absolute value of negative dispersion generated in said negative dispersion device for said light pulse be equal or approximately equal to the absolute value of positive dispersion generated within the light pulse traveling path to said object in said optical turbid medium for said light pulse, but with the opposite sign.

9. The apparatus of claim 1, wherein said inner light layer formed in said optical turbid medium is plane, or cylindrical, or spherical layer, or further becoming line or point via focusing by lens(es) in said set of optical components.

10. The apparatus of claim 1, in the wherein said apparatus, the optical components including triangular components, lenses, or prism(s), or beam splitter(s) are totally or partially made of the same material as the turbid medium containing said object, or are totally or partially made of the material which has the same or approximately same dispersion property as that of the turbid medium containing said object.

11. The apparatus of claim 1, wherein said processing distance adjuster comprising a hollow optical fiber or a hollow thin tube is that said hollow optical fiber or hollow thin tube is filled with the corresponding turbid medium containing said object, or is filled with the material which has the same or approximately same dispersion property as that of the corresponding turbid medium containing said object.

12. The apparatus of claim 1, wherein said processing distance adjuster changes the processing distance to said object in said optical turbid medium is by changing an additional distance outside said optical turbid medium.

13. The apparatus of claim 1, wherein said processing object in optical turbid medium is performed repeatedly.

14. The apparatus of claim 1, wherein said light pulse is reflected from said object and becomes signal light pulse, by utilizing said set of optical components, the said signal light pulse goes back in reverse order through said optical turbid medium, said processing distance adjuster, said negative dispersion device and finally is received by an image receiver for image-guided processing.

15. The apparatus of claim 14, wherein said image-guided processing is performed repeatedly.
